(19) United States
(12) Patent Application Publication
(10) Pub. No.: US 2013/0281311 A1
(43) Pub. Date: Oct. 24, 2013

(54) SPECIFIC BIOMARKER FOR IDENTIFICATON OF EXPOSURE TO PROPIONALDEHYDE AND THE METHOD OF IDENTIFICATION USING THE SAME

(71) Applicant: KOREA INSTITUTE OF SCIENCE AND TECHNOLOGY, Seoul (KR)

(72) Inventors: Jae Chun Ryu, Seoul (KR); Mee Song, Seoul (KR); Ji-seong Yoon, Seoul (KR); Hyo Sun Lee, Seoul (KR); Wooin Ryu, Seoul (KR); Chan Young Shin, Seoul (KR)

(73) Assignee: KOREA INSTITUTE OF SCIENCE AND TECHNOLOGY, Seoul (KR)

(21) Appl. No.: 13/689,042

(22) Filed: Nov. 29, 2012

(30) Foreign Application Priority Data
Apr. 18, 2012 (KR) 10-2012-0040407

(51) Int. Cl. C12Q 1/68

(52) U.S. Cl. CPC C12Q 1/6813; C12Q 1/686
USPC 506/9; 435/6.12

(57) ABSTRACT

The present invention relates to a biomarker for the identification of specific exposure to propionaldehyde which is one of volatile organic compounds exposed in the environment, and a method for the identification of specific exposure to propionaldehyde using the same, precisely a biomarker which is up-regulated or down-regulated specifically by propionaldehyde and a method for the identification of specific exposure to propionaldehyde using the biomarker. The biomarker of the present invention is the reacted genes selected by using DNA microarray chip, which can be effectively used for the monitoring and evaluation of propionaldehyde contamination in the environment samples and at the same time as a tool for the investigation of the toxic mechanism induced specifically by propionaldehyde.

### CROSS-REFERENCES TO RELATED APPLICATION

This patent application claims the benefit of priority under 35 U.S.C. §119 from Korean Patent Application Nos. 10-2012-0040407 filed on Apr. 18, 2012 the contents of which are incorporated herein by reference.

### BACKGROUND OF THE INVENTION

1. Field of the Invention

The present invention relates to a specific biomarker for the identification of exposure to propionaldehyde and a method for the identification of such exposure using the same, more precisely a biomarker which is specifically up-regulated or down-regulated by propionaldehyde and a method for the identification of specific exposure to propionaldehyde using the said biomarker.

2. Description of the Related Art

Propionaldehyde, known as a colorless material generating very nasty smell, has been wildly used not only for the production of propion acid, polyvinyl, and other plastics, but also for rubber synthesis, disinfection and antiseptic effect. Propionaldehyde is emitted in the environment primarily by combustion of wood, gasoline, diesel fuel, and polyethylene. Human body can also be exposed on propionaldehyde through smoking and by taking food additives and spices as well. The distribution rate of propionaldehyde in the environment is the third highest next to formaldehyde and acetaldehyde, among many aldehydes (EPA. 2008).

It has been reported that propionaldehyde exposure to human body mainly causes respiratory diseases and cardiovascular diseases. Propionaldehyde is metabolized by aldehyde dehyderonase. According to the previous reports, propionaldehyde exposure to human body causes mutations of ALDH and ALDH2 genes (Drug Chem Toxicol 20(3):173-187, 1997; Drug Metab Dispos 30(1):69-73, 2002). It has been also reported that propionaldehyde causes vacuolization and atrophy in olfactory epithelial cells even at a low concentration confirmed by the evaluation test of in vivo exposure of propionaldehyde in rats. The above results suggest that propionaldehyde is closely related with respiratory diseases.

The volatile organic compounds flowing through bloodstream affect the lung by diffusion of lung sac membrane. Hexane, methylpentan, isopropene, and benzene have been used as markers for the respiratory diseases (J Vet Sci 5(1):11-18, 2004). Recently, a simple diagnostic method for lung cancer has been developed based on the results of exhaled breath analysis on volatile organic compounds. Among the volatile organic compounds, aldehydes are the most representative materials commonly found in lung cancer patients (J Chromatogr B Analyt Technol Biomed Life Sci. 878(27):2643-2651, 2010). Therefore, the aldehyde specific biomarker, especially the propionaldehyde specific biomarker can be effectively used for the screening of pulmonary disease and propionaldehyde exposure in the environment.

Despite the hazard in human, risk assessment data of propionaldehyde are not enough and the methods for the screening of the propionaldehyde exposure are limited to a few classical methods such as GC-MS (Gas Chromatography-Mass Spectrometer) or HPLC (High Performance Liquid Chromatography). GC-MS or HPLC enables quantitative analysis but proper conditions have to be set up first and expensive equipments are required for the analysis. Thus, it is important to establish molecular index for the screening of toxicity and specific gene expression in human via faster and simpler methods such as real-time RT-PCR (real-time reverse transcript polymerase chain reaction) using primers or DNA microarray chip for the fast risk assessment, and to control and manage propionaldehyde exposure.

Genome sequencing project has been completed with 6 species of mammals and 292 species of microorganisms, which has been reported to NCBI (National Center for Biotechnology Information). Based on the huge amount of data obtained thereby, genome-wide expression has been studied to understand the functions of genes. DNA microarray analysis has been performed to analyze thousands of genes at a time (Proc. Natl. Acad. Sci. USA 93:10614-10619, 1996).

Microarray indicates the glass board on which many sets of cDNA (complementary DNA) or 20-25 base pair long oligonucleotides are integrated. CDNA microarray is now produced by ink jetting or by fixing cDNA mechanically on the chip in laboratories of schools or companies including Agilent and Genomic Solutions, etc. (J. Am. Acad. Dermatol. 51:681-692, 2004). Oligonucleotide microarray is produced by direct synthesis on the chip using photolithography by Affymetrix Co., or via fixation of synthesized oligonucleotides by Agilent Co. (J. Am. Acad. Dermatol. 51:681-692, 2004).

To analyze gene expression, RNA is first extracted from tissues or cells, followed by hybridization with oligonucleotides on DNA microarray. The obtained RNA is labeled with fluorescein or isotope, which is then converted into cDNA. In oligo microarray, each of the control group and the experimental group is labeled with a different fluorescent materials (ex: Cye3 and Cye5) but hybridization is induced on the same chip simultaneously. Optical image is scanned to measure fluorescence signal. Gene expression is determined by comparing the two different fluorescence signals (Genomics Proteomics I: 1-10, 2002). The cooperation with toxicogenomics, the most recent technology using DNA microarray, enables high throughput quantitative analysis and expression pattern analysis of genes expressed in a specific tissue or cell line triggered by every chemical including not only drugs and new drug candidates but also representative environmental contaminants. Thus, specific genes that are involved in side effects of drugs and adverse actions of environmental contaminants can be identified by analyzing specific gene expression in specific cells. Accordingly, adverse actions of environmental contaminants and molecular mechanisms related to functions and side effects of drugs can be understood and further screening and identification of such material that causes toxicity and side effects can be achieved.

The present inventors observed and analyzed gene expression profiles affected by propionaldehyde in A459 cell line, the human lung cancer tissue derived cell line, by using oligo microarray on which 42,000 human genes are integrated. As a result, the present inventors completed this invention by establishing a biomarker that is able to detect propionaldehyde specifically by using a gene up-regulated or down-regulated specifically by propionaldehyde among many other environmental materials and a method for the identification of specific exposure to propionaldehyde using the said biomarker.

### SUMMARY OF THE INVENTION

It is an object of the present invention to provide a biomarker that is up-regulated or down-regulated specifically by propionaldehyde exposure and a method for the identification of specific exposure to propionaldehyde using the said biomarker.

To achieve the above object, the present invention provides a biomarker for the identification of propionaldehyde specific exposure whose expression is specifically changed by exposure to propionaldehyde.

The present invention also provides a DNA microarray chip for the identification of propionaldehyde specific exposure, on which nucleic acid sequences or their complementary strand molecules of one or more genes selected from the below group are integrated:

Genebank accession number NM_{—}000029 (AGT, angiotensinogen; SEQ. ID. NO: 11), Genebank accession number NM_{—}057159 (LPAR1, lysophosphatidic acid receptor 1; SEQ. ID. NO: 12), Genebank accession number NM_{—}003004 (SECTM1, secreted and transmembrane 1; SEQ. ID. NO: 13), Genebank accession number NM_{—}003810 (TNFSF10, tumor necrosis factor (ligand) superfamily, member 10; SEQ. ID. NO: 14), and Genebank accession number NM_{—}002133 (HMOX1, heme oxygenase 1; SEQ. ID. NO: 15).

The present invention further provides A method for the identification of exposure to propionaldehyde comprising the following steps:

1) measuring expression levels of genes of:

Genebank accession number NM_{—}000029 (AGT, angiotensinogen; SEQ. ID. NO: 11), Genebank accession number NM_{—}057159 (LPAR1, lysophosphatidic acid receptor 1; SEQ. ID. NO: 12), Genebank accession number NM_{—}003004 (SECTM1, secreted and transmembrane 1; SEQ. ID. NO: 13), Genebank accession number NM_{—}003810 (TNFSF10, tumor necrosis factor (ligand) superfamily, member 10; SEQ. ID. NO: 14), and Genebank accession number NM_{—}002133 (HMOX1, heme oxygenase 1; SEQ. ID. NO: 15), on somatic cells separated from both an experimental group suspected with propionaldehyde exposure and a normal control group;

2) screening a subject with increased or decreased expression level by comparing the expression level of the experimental group of step 1) with that of the control group; and

3) determining the screened object of step 2) to be exposed to propionaldehyde.

The present invention further provides a method for the identification of exposure to propionaldehyde comprising the following steps:

1) extracting RNA from somatic cells obtained from both the experimental group highly suspected with propionaldehyde exposure and the normal control group;

2) synthesizing cDNA from the RNA extracted from both the experimental group and the control group of step 1), followed by labeling with different fluorescent materials;

3) hybridizing each cDNA labeled with different fluorescent materials of step 2) with the DNA microarray chip of the present invention;

4) analyzing the reacted DNA microarray chip; and

5) confirming the exposure to propionaldehyde by comparing the expressions of the genes integrated on the DNA microarray chip of the present invention with those of the control based on the data analyzed.

The present invention also provides a method for the identification of exposure to propionaldehyde comprising the following steps:

1) extracting RNA from somatic cells obtained from both the experimental group highly suspected with propionaldehyde exposure and the normal control group;

2) performing real-time RT-PCR (real-time reverse transcript polymerase chain reaction) with the obtained RNA using the primer sets complementary to the below genes and able to amplify them as well:

Genebank accession number NM_{—}000029 (AGT, angiotensinogen; SEQ. ID. NO: 11), Genebank accession number NM_{—}057159 (LPAR1, lysophosphatidic acid receptor 1; SEQ. ID. NO: 12), Genebank accession number NM_{—}003004 (SECTM1, secreted and transmembrane 1; SEQ. ID. NO: 13), Genebank accession number NM_{—}003810 (TNFSF10, tumor necrosis factor (ligand) superfamily, member 10; SEQ. ID. NO: 14), and Genebank accession number NM_{—}002133 (HMOX1, heme oxygenase 1; SEQ. ID. NO: 15); and

3) confirming the expression by comparing the gene product obtained in step 2) with that of the control.

The present invention also provides a kit for the identification of exposure to propionaldehyde comprising the DNA microarray chip of the present invention.

In addition, the present invention provides a kit for the identification of exposure to propionaldehyde comprising the primer set that is complementary to each of the below genes and is able to amplify each of them as well:

Genebank accession number NM_{—}000029 (AGT, angiotensinogen), Genebank accession number NM_{—}057159 (LPAR1, lysophosphatidic acid receptor 1; SEQ. ID. NO: 12), Genebank accession number NM_{—}003004 (SECTM1, secreted and transmembrane 1; SEQ. ID. NO: 13), Genebank accession number NM_{—}003810 (TNFSF10, tumor necrosis factor (ligand) superfamily, member 10; SEQ. ID. NO: 14), and Genebank accession number NM_{—}002133 (HMOX1, heme oxygenase 1; SEQ. ID. NO: 15).

### Advantageous Effect

As explained hereinbefore, the specific biomarker for the identification of exposure to propionaldehyde and the method for the identification of such exposure using the same of the present invention are very useful for the monitoring propionaldehyde using the reactive gene selected by using DNA microarray chip as a biomarker and for the risk assessment and also as a tool to explain the mechanism of propionaldehyde specific toxicity.

### BRIEF DESCRIPTION OF THE DRAWINGS

The application of the preferred embodiments of the present invention is best understood with reference to the accompanying drawings, wherein:

FIG. 1 is a graph illustrating the cytotoxicity of propionaldehyde in the human lung cancer tissue derived cell line.

FIG. 2 is a graph illustrating the result of the gene expression analysis with the human lung cancer tissue derived cell line treated with propionaldehyde by using microarray chip:

Middle line (M=0): R (fluorescence strength of the experimental group)=G (fluorescence strength of the control group);

M=1: Probes showing 1.5 times higher R (fluorescence strength of the experimental group) than G (fluorescence strength of the control) are distributed. Higher spots indicate the distribution of probes having 1.5 fold stronger signal intensity.

FIG. 3 is a graph illustrating the comparison of gene expression profiles obtained by treating butylaldehyde, valeradehyde, hexanal, heptanal, octanal, and propionaldehyde to select those genes showing up-regulation or down-regulation specifically by propionaldehyde.

### DESCRIPTION OF THE PREFERRED EMBODIMENTS

Hereinafter, the present invention is described in detail.

The present invention provides a biomarker for the identification of propionaldehyde specific exposure whose expression is specifically changed by exposure to propionaldehyde.

The said biomarker is the genes whose expressions are 1.5 fold increased or decreased by propionaldehyde. Particularly the biomarker is composed of 5 kinds of genes whose expressions are specifically changed by propionaldehyde.

The biomarker whose expression is specifically changed by propionaldehyde exposure is preferably selected from the group composed of as followings, but not always limited thereto:

Genebank accession number NM_{—}000029 (AGT, angiotensinogen; SEQ. ID. NO: 11), Genebank accession number NM_{—}057159 (LPAR1, lysophosphatidic acid receptor 1; SEQ. ID. NO: 12), Genebank accession number NM_{—}003004 (SECTM1, secreted and transmembrane 1; SEQ. ID. NO: 13), Genebank accession number NM_{—}003810 (TNFSF10, tumor necrosis factor (ligand) superfamily, member 10; SEQ. ID. NO: 14), and Genebank accession number NM_{—}002133 (HMOX1, heme oxygenase 1; SEQ. ID. NO: 15).

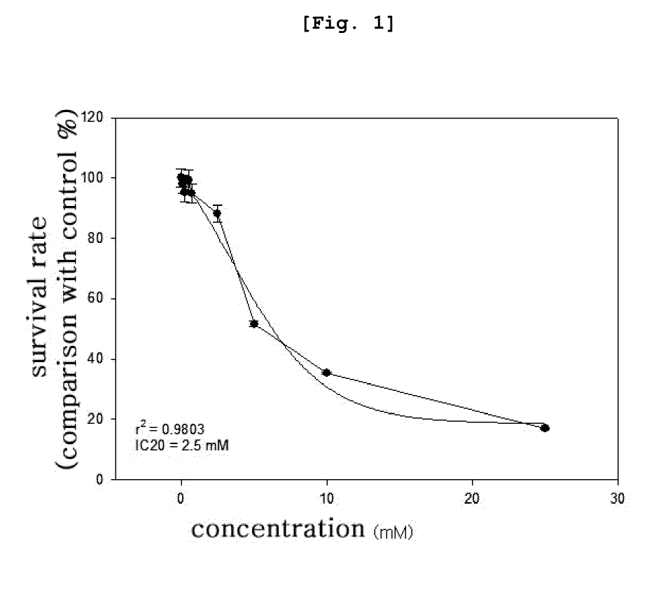

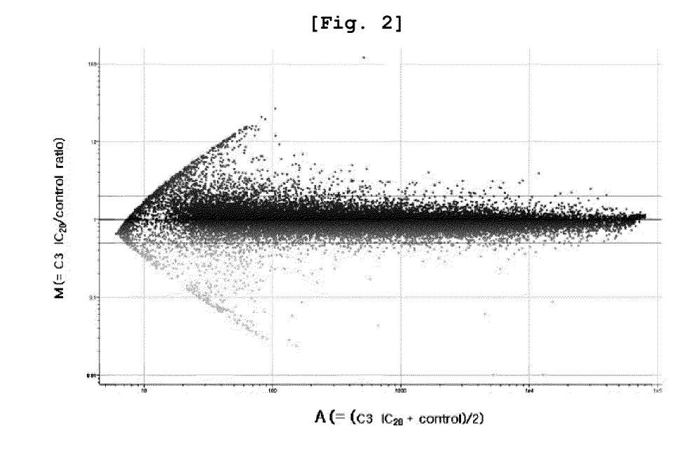

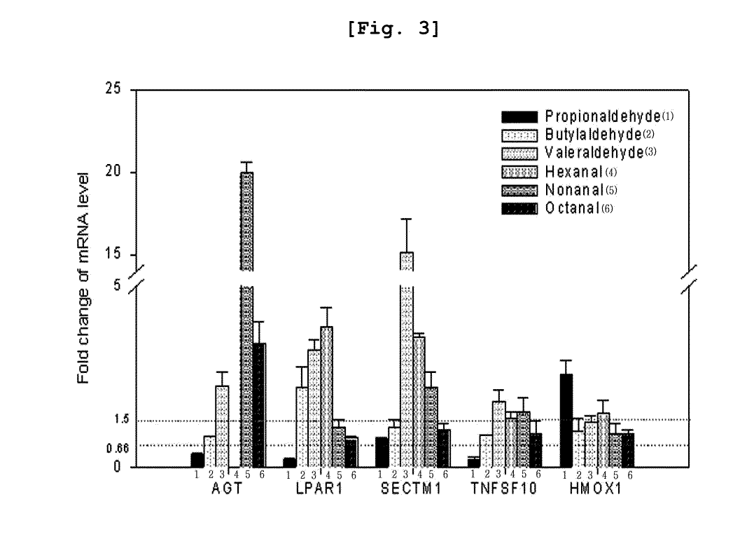

In a preferred embodiment of the present invention, to screen a biomarker for the identification of propionaldehyde specific exposure, the present inventors treated propionaldehyde to the human lung cancer tissue derived cell line A549 and then investigated cytotoxicity therein. As a result, it was confirmed that the said propionaldehyde had cytotoxicity in the human lung cancer tissue derived cell line (see FIG. 1). Based on the result of that experiment, the concentration of propionaldehyde was determined. Propionaldehyde was treated to the human lung cancer tissue derived cell line at the determined concentration. From the cell line treated with propionaldehyde, mRNA was extracted, followed by synthesis of cDNA which was labeled with fluorescein (Cy5). The control not treated with propionaldehyde was labeled with Cy3. The fluorescein-labeled cDNA was hybridized with 8×60 k oligomicroarray chip [Human whole genome oligo microarray (Agilent, USA)], followed by scanning fluorescence image to analyze gene expression patterns (see FIG. 2). When the ratio of Cy5 to Cy3 was higher than 1.5, the gene was regarded as the one whose expression is increased. When the ratio was lower than 0.66, the gene was regarded as the one whose expression is decreased. From the result of the analysis, it was confirmed that the gene whose expression was increased took 2.12% (900 genes out of 42,405) and the gene whose expression was decreased took 3.59% (1,522 genes out of 42,405). Based on the biological functions of those genes showing expressional changes, 5 kinds of genes belonging to protein kinase cascade were selected. 5 kinds of genes whose expressions were increased or decreased specifically by propionaldehyde alone among 5 kinds of aldehydes (butylaldehyde, valeradehyde, hexanal, heptanal, octanal, and propionaldehyde) were selected by using real-time quantitative PCR (see FIG. 3, Table 3, and Table 4). These genes were reported to be involved in the development of lung toxicity related diseases triggered by other chemicals in previous studies, but there have been no reports saying that the treatment of propionaldehyde on them causes toxicity in human lung cancer tissue derived cells.

The present invention also provides a DNA microarray chip for the identification of propionaldehyde specific exposure, on which nucleic acid sequences or their complementary strand molecules of one or more genes selected from the below group are integrated:

Genebank accession number NM_{—}000029 (AGT, angiotensinogen), Genebank accession number NM_{—}057159 (LPAR1, lysophosphatidic acid receptor 1; SEQ. ID. NO: 12), Genebank accession number NM_{—}003004 (SECTM1, secreted and transmembrane 1; SEQ. ID. NO: 13), Genebank accession number NM_{—}003810 (TNFSF10, tumor necrosis factor (ligand) superfamily, member 10; SEQ. ID. NO: 14), and Genebank accession number NM_{—}002133 (HMOX1, heme oxygenase 1; SEQ. ID. NO: 15).

The DNA microarray chip for the identification of propionaldehyde specific exposure of the present invention can be prepared by the method well known to those in the art. Precisely, the method for the preparation of the said microarray chip is as follows. To fix the screened biomarker to be used as a probe DNA molecule on DNA chip board, micropipetting based on piezolelectric method or pin spotter is preferably used, but not always limited thereto. In a preferred embodiment of the present invention, pin-spotter microarray was used. The DNA microarray chip board is preferably coated with one of active groups selected from the group consisting of amino-silane, poly-L-lysine, and aldehyde, but not always limited thereto. The board is also selected from the group consisting of slide glass, plastic, metal, silicon, nylon membrane, and nitrocellulose membrane, but not always limited thereto. In a preferred embodiment of the present invention, amino-silane coated slide glass was used as the board.

The present invention also provides a method for the identification of propionaldehyde specific exposure using the biomarker of the present invention.

The present invention provides a method for the identification of exposure to propionaldehyde comprising the following steps:

1) measuring expression levels of genes of:

Genebank accession number NM_{—}000029 (AGT, angiotensinogen; SEQ. ID. NO: 11), Genebank accession number NM_{—}057159 (LPAR1, lysophosphatidic acid receptor 1; SEQ. ID. NO: 12), Genebank accession number NM_{—}003004 (SECTM1, secreted and transmembrane 1; SEQ. ID. NO: 13), Genebank accession number NM_{—}003810 (TNFSF10, tumor necrosis factor (ligand) superfamily, member 10; SEQ. ID. NO: 14), and Genebank accession number NM_{—}002133 (HMOX1, heme oxygenase 1; SEQ. ID. NO: 15), on somatic cells separated from both an experimental group suspected with propionaldehyde exposure and a normal control group;

2) screening a subject with increased or decreased expression level by comparing the expression level of the experimental group of step 1) with that of the control group; and

3) determining the screened object of step 2) to be exposed to propionaldehyde.

In this identification method, the somatic cell of step 1) is preferably A549, the human lung cancer tissue derived cell line, but not always limited thereto, and any human lung cell or human lung cancer cell and tissue derived cell can be used.

In the identification method, the comparing the expression level in step 1) is performed at the level gene or protein. At this time. The gene level can be performed by RT-PCR, competitive RT-PCR, real-time RT-PCR, RNase protection assay, Northern blotting, and DNA chip. AND, the protein level can be performed by microarray or ELISA.

In the identification method, the expression level of the genes of Genebank accession number NM_{—}000029 (AGT, angiotensinogen; SEQ. ID. NO: 11), Genebank accession number NM_{—}057159 (LPAR1, lysophosphatidic acid receptor 1; SEQ. ID. NO: 12), Genebank accession number NM_{—}003004 (SECTM1, secreted and transmembrane 1; SEQ. ID. NO: 13), and Genebank accession number NM_{—}003810 (TNFSF10, tumor necrosis factor (ligand) superfamily, member 10; SEQ. ID. NO: 14), is down-regulated when exposed to propionaldehyde.

In the identification method, the expression level of the gene of Genebank accession number NM_{—}002133 (HMOX1, hemeoxygenase 1; SEQ. ID. NO: 15) is up-regulated when exposed to propionaldehyde.

The present invention provides a method for the identification of specific exposure to propionaldehyde comprising the following steps:

1) extracting RNA from somatic cells obtained from both the experimental group highly suspected with propionaldehyde exposure and the normal control group;

2) synthesizing cDNA from the RNA extracted from both the experimental group and the control group of step 1), followed by labeling with different fluorescent materials;

3) hybridizing each cDNA labeled with different fluorescent materials of step 2) with the DNA microarray chip of the present invention;

4) analyzing the reacted DNA microarray chip; and

5) confirming the exposure to propionaldehyde by comparing the expressions of the genes integrated on the DNA microarray chip of the present invention with those of the control based on the data analyzed.

In this identification method, the somatic cell of step 1) is preferably A549, the human lung cancer tissue derived cell line, but not always limited thereto, and any human lung cell or human lung cancer cell and tissue derived cell can be used.

In the identification method, the fluorescent material of step 3) is preferably selected from the group consisting of Cy3, Cy5, poly L-lysine-fluorescein isothiocyanate (FITC), rhodamine-B-isothiocyanate (RITC), and rhodamine, but not always limited thereto, and any fluorescent material that is well known to those in the art can be used.

In the identification method of the present invention, the DNA microarray chip of step 4) is preferably whole human genome oligo microarray chip (Agilent, USA), but not always limited thereto, and any microarray chip loaded with gene demonstrating up-regulation or down-regulation (see Table 4), among human genome, can be used. The DNA microarray chip constructed by the present inventors is more preferred. In the analyzing method of step 4), Agilent Feature Extraction 10.7.3.1 (Agilent technologies, CA, USA), or Agilent GeneSpring GX 11.5.1 (Agilent technologies, CA, USA) is preferably used, but not always limited thereto, and any software for such analysis known to those in the art can be used.

The present invention also provides a method for the identification of exposure to propionaldehyde comprising the following steps:

1) extracting RNA from somatic cells obtained from both the experimental group highly suspected with propionaldehyde exposure and the normal control group;

2) performing real-time RT-PCR (real-time reverse transcript polymerase chain reaction) with the obtained RNA using the primer sets complementary to the below genes and able to amplify them as well:

Genebank accession number NM_{—}000029 (AGT, angiotensinogen; SEQ. ID. NO: 11), Genebank accession number NM_{—}057159 (LPAR1, lysophosphatidic acid receptor 1; SEQ. ID. NO: 12), Genebank accession number NM_{—}003004 (SECTM1, secreted and transmembrane 1; SEQ. ID. NO: 13), Genebank accession number NM_{—}003810 (TNFSF10, tumor necrosis factor (ligand) superfamily, member 10; SEQ. ID. NO: 14), and Genebank accession number NM_{—}002133 (HMOX1, heme oxygenase 1; SEQ. ID. NO: 15); and 3) confirming the expression by comparing the gene product obtained in step 2) with that of the control.

The primer set of step 2) is preferably composed of 18-30 mer long forward primer and reverse primer to amplify the gene of step 2), and more preferably selected from the group consisting of the following primer set 1 primer set 5, but not always limited thereto:

Primer set 1: forward primer represented by SEQ. ID. NO: 1 and reverse primer represented by SEQ. ID. NO: 2;

Primer set 2: forward primer represented by SEQ. ID. NO: 3 and reverse primer represented by SEQ. ID. NO: 4;

Primer set 3: forward primer represented by SEQ. ID. NO: 5 and reverse primer represented by SEQ. ID. NO: 6;

Primer set 4: forward primer represented by SEQ. ID. NO: 7 and reverse primer represented by SEQ. ID. NO: 8; and

Primer set 5: forward primer represented by SEQ. ID. NO: 9 and reverse primer represented by SEQ. ID. NO: 10.

Therefore, the biomarker of the present invention can be effectively used for the monitoring and evaluation of propionaldehyde contamination in environmental examples because the expression of the marker is specifically increased or decreased by propionaldehyde.

The present invention also provides a kit for the identification of specific exposure to propionaldehyde comprising the DNA microarray chip constructed in this invention.

The kit preferably contains human somatic cells additionally, but not always limited thereto.

The said human somatic cell is preferably A549, but not always limited thereto, and any human lung cell or human lung cancer cell and tissue derived cell can be used.

The kit can additionally include fluorescent material which is preferably selected from the group consisting of streptavidin-like phosphatase conjugate, chemifluorescence, and chemiluminescent, but not always limited thereto. In a preferred embodiment of the present invention, Cy3 and Cy5 were used.

The kit can additionally include reaction reagent which is exemplified by buffer used for hybridization, reverse transcriptase for cDNA synthesis from RNA, cNTPs and rNTP (premix type or separately supplied type), labeling reagent such as chemical inducer of fluorescent dye, and washing buffer, but not always limited thereto, and any reaction reagent required for DNA microarray chip hybridization known to those in the art can be included.

The biomarker of the present invention is up-regulated or down-regulated specifically by propionaldehyde, so that it can be effectively used for the monitoring and evaluation of propionaldehyde contamination in the environment samples and as a tool to explain the mechanism of propionaldehyde specific toxicity.

In addition, the present invention provides a kit for the identification of exposure to propionaldehyde comprising the primer set that is complementary to each of the below genes and is able to amplify each of them as well:

Genebank accession number NM_{—}000029 (AGT, angiotensinogen; SEQ. ID. NO: 11), Genebank accession number NM_{—}057159 (LPAR1, lysophosphatidic acid receptor 1; SEQ. ID. NO: 12), Genebank accession number NM_{—}003004 (SECTM1, secreted and transmembrane 1; SEQ. ID. NO: 13), Genebank accession number NM_{—}003810 (TNFSF10, tumor necrosis factor (ligand) superfamily, member 10; SEQ. ID. NO: 14), and Genebank accession number NM_{—}002133 (HMOX1, heme oxygenase 1; SEQ. ID. NO: 15).

The primer set included in the said kit is preferably selected from the group consisting of the following primer set 1˜primer set 5, but not always limited thereto. Any forward primer and reverse primer set in the length of 15˜50 mer, more preferably in the length of 15˜30 mer, and most preferably in the length of 18˜25 mer to produce the amplified product of the biomarker gene to be 100˜300 bp long can be used.

Primer set 1: forward primer represented by SEQ. ID. NO: 1 and reverse primer represented by SEQ. ID. NO: 2;

Primer set 2: forward primer represented by SEQ. ID. NO: 3 and reverse primer represented by SEQ. ID. NO: 4;

Primer set 3: forward primer represented by SEQ. ID. NO: 5 and reverse primer represented by SEQ. ID. NO: 6;

Primer set 4: forward primer represented by SEQ. ID. NO: 7 and reverse primer represented by SEQ. ID. NO: 8; and

Primer set 5: forward primer represented by SEQ. ID. NO: 9 and reverse primer represented by SEQ. ID. NO: 10.

The kit for the identification preferably contains human somatic cells additionally, but not always limited thereto.

The said human somatic cell is preferably A549, but not always limited thereto, and any human lung cell or human lung cancer cell and tissue derived cell can be used.

The kit can additionally include reaction reagent which is exemplified by reverse transcriptase for cDNA synthesis from RNA, cNTPs and rNTP (premix type or separately supplied type), labeling reagent such as chemical inducer of fluorescent dye, and washing buffer, but not always limited thereto, and any reaction reagent required for RT-PCR known to those in the art can be included.

Practical and presently preferred embodiments of the present invention are illustrative as shown in the following Examples, Experimental Examples and Manufacturing Examples.

However, it will be appreciated that those skilled in the art, on consideration of this disclosure, may make modifications and improvements within the spirit and scope of the present invention.

### Example 1

### Cell Culture and Chemical Treatment

### <1-1> Cell Culture

A549 cells (Korean Cell Line Bank), the human lung cancer tissue derived cell line, were cultured in 100 mm dish containing RPMI (Gibro-BRL, USA) supplemented with 10% FBS until the confluency reached 80%. The present inventors selected propionaldehyde, one of aldehydes among many volatile organic compounds exposed in environment, as a target material based on the previous studies and reports, and then dissolved in DMSO (dimethyl sulfoxide). The concentration of vehicle was up to 0.1% in every experiment.

### <1-2> Cytotoxicity Test (MTT Assay) and Chemical Treatment

MTT assay was performed with A549 cell line according to the method of Mossman, et al (J. Immunol. Methods, 65, 55-63, 1983).

Particularly, the cells were distributed in 24-well plate (3.5×10⁴ cells/well) containing RPMI (Gibro-BRL, USA) and then treated with propionaldehyde dissolved in DMSO. 48 hours later, 5 mg/ml of MTT (3-4,5-dimethylthiazol-2,5-diphenyltetra zolium bromide) was added thereto, followed by culture at 37° C. for 3 hours. Then, the medium was discarded and the formed formazan crystal was dissolved in 500 ml of DMSO, which was aliquoted in 96-well plate. OD₅₄₀ was measured. As shown in FIG. 1, cytotoxicity in A549 by propionaldehyde was measured.

As a result, as shown in FIG. 1, IC₂₀ (the concentration showing 20% survival rate) was 2.5 mM. Based on this result, the following microarray experiment was performed (FIG. 1).

### Example 2

### Microarray Experiment

### <2-1> Separation of Target RNA and Fluorescein Labeling

A549 cells were distributed in 6-well plate at the density of 25×10⁴ cells/ml, to which propionaldehyde was treated for 48 hours at the concentration determined in Example <1-2>. Total RNA was extracted from the cells by using trizol reagent according to the manufacturer's protocol (Invitrogen life technologies, USA), followed by purification by using RNease mini kit (Qiagen, USA). Genomic DNA was eliminated by using RNase-free DNase set (Qiagen, USA) during the RNA purification. The amount of total RNA was measured with spectrophotometer, and the concentration was confirmed by ND-1000 Spectrophotometer (Thermo Fisher Scientific Inc., USA) and Agilent 2100 Bioanalyzer (Agilent).

<2-2> Preparation of Labeled cDNA

For oligomicroarray analysis, cDNA was synthesized by using the total RNA obtained from the experimental group treated with propionaldehyde prepared in Example <2-1>. 30 μg of the obtained total RNA and 2 μg of oligo (dT) primer (1 μg/μl) were mixed together, followed by reaction at 65° C. for 10 minutes, which was transferred into ice for annealing. Upon completion of annealing, reagents were added thereto as shown in Table 1 to induce reverse transcription of the annealed RNA. The total RNA extracted from the control group A549 cells was labeled with Cy3-dUTP (green). The total RNA extracted from the experimental group A549 cells treated with propionaldehyde was labeled with Cy5-dUTP (red). At that time, the two samples were mixed and purified by using Microcon YM-30 column (Millipore, USA).

TABLE 1

| Composition | Volume (μl) |
|---|---|
| 5X first strand buffer | 6 |
| dNTPs | 0.6 |
| 0.1M DDT | 3 |
| SuperScript II enzyme | 3 |
| Cy-3 or Cy-5 dUTP | 2 |

### <2-3> Hybridization

Hybridization and washing processes were performed according to the protocol provided by Ebiogen Inc.

Particularly, transcription master-mix was prepared as shown in Table 2, followed by reaction at 40° C. for 2 hours. Labeled cRNA was purified. 600 ng of the purified cRNA was reacted at 60° C. for 30 minutes for fragmentation. The prepared cRNA was mixed with 2× GEx Hybridization Buffer HI-RPM. After well mixing, the mixture was loaded on chip, followed by hybridization in the oven at 65° C. for 17 hours. 17 hours later, the chip was washed with GE Wash Buffer 1 for 1 minute and with GE Wash Buffer 2 for 1 minute. Centrifugation was performed with the chip at 800 rpm for 3 minutes, followed by drying thereof.

TABLE 2

| Component | Volume (μl) per reaction |
|---|---|
| nuclease-free water | 0.75 |
| 5X Transcription Buffer | 3.2 |
| 0.1M DTT | 0.6 |
| TP mix | 1 |
| T7 RNA Polymerase Blend | 0.21 |
| Cyanine 3-CTP | 0.24 |

### <2-4> Obtainment of Fluorescence Image

Hybridization images on the slide were scanned with Agilent C scanner (Agilent technologies, CA, USA). At that time, the green fluorescent image indicated the activity of gene expressed specifically in the control group, while the red fluorescent image indicated the activity of gene expressed specifically in the experimental group. In the meantime, the yellow fluorescent image indicated that there was no big difference in the expression between those genes respectively presented by red and green. To obtain gene expression rate, the scanned images were analyzed by using Agilent Feature Extraction 10.7.3.1 (Agilent technologies, CA, USA). The extracted data proceeded to normalization by using Agilent GeneSpring GX 11.5.1 (Agilent technologies, CA, USA) to analyze gene expression pattern of each gene. The marker gene for propionaldehyde was selected from the obtained data.

As a result, as shown in FIG. 2, among 42,000 genes existed on the oligo chip, the genes showing at least 1.5 fold higher expression (Cy5/Cy3 ratio) by propionaldehyde took 2.12% (900 genes out of 42,405 genes) and the genes showing lower expression took 3.59% (1,522 genes out of 42,405 genes) (FIG. 2).

To select those genes whose expressions were specifically increased or decreased by propionaldehyde, gene expression profiles obtained by treating 5 kinds of aldehydes, other than propionaldehyde, such as butylaldehyde, valeraldehyde, hexanal, heptanal, and octanal, shown in Table 3, were compared and analyzed.

As a result, as shown in FIG. 3, 5 kinds of genes whose expressions were increased or decreased specifically by propionaldehyde were selected (FIG. 3 and Table 4). In addition, there have been no reports saying that the genes are related to toxicity in human lung cancer tissue derived cells when propionaldehyde was treated.

TABLE 3

| | Name | Conc. |
|---|---|---|
| Aldehydes | Butylaldehyde | 4.6 mM |
| | Valeraldehyde | 3.4 mM |
| | Hexanal | 1.6 mM |
| | Heptanal | 0.6 mM |
| | Octanal | 0.58 mM |

TABLE 4

| | | | Median ratio of |
|---|---|---|---|
| | | | microarray by |
| Accession | | | propionaldehyde |
| Number | Abbreviation | Gene Name | exposure |
| NM_000029; | AGT | Angiotensinogen | 0.55 |
| SEQ. ID. | | | |
| NO: 11 | | | |
| NM_057159; | LPAR1 | Lysophosphatidic | 0.62 |
| SEQ. ID. | | acid receptor 1 | |
| NO: 12 | | | |
| NM_003004; | SECTM1 | Secreted and | 0.63 |
| SEQ. ID. | | transmembrane 1 | |
| NO: 13 | | | |
| NM_003810; | TNFSF10 | Tumor necrosis | 0.65 |
| SEQ. ID. | | factor (ligand) | |
| NO: 14 | | superfamily, member | |
| | | 10 | |
| NM_002133; | HMOX1 | Heme oxygenase 1 | 1.52 |
| SEQ. ID. | | | |
| NO: 15 | | | |

### Example 3

### Real Time RT-PCR

To investigate and quantify the expressions of 5 different genes confirmed to be expressed specifically by propionaldehyde [Genebank accession number NM_{—}000029 (AGT, angiotensinogen; SEQ. ID. NO: 11), Genebank accession number NM_{—}057159 (LPAR1, lysophosphatidic acid receptor 1; SEQ. ID. NO: 12), Genebank accession number NM_{—}003004 (SECTM1, secreted and transmembrane 1; SEQ. ID. NO: 13), Genebank accession number NM_{—}003810 (TNFSF10, tumor necrosis factor (ligand) superfamily, member 10; SEQ. ID. NO: 14), and Genebank accession number NM_{—}002133 (HMOX1, heme oxygenase 1; SEQ. ID. NO: 15)], selected in Example 2 among many genes demonstrating up-regulation or down-regulation specifically by propionaldehyde, quantitative real-time RT-PCR was performed using My IQ real-time PCR (Bio-rad, USA). At that time, to confirm the propionaldehyde specific expression changes, the said genes were investigated with their expression patterns using the additional 5 aldehydes listed in Table 3.

Particularly, cDNA was synthesized by performing reverse transcription by using oligo dT primer and Superscript kit (Omniscipt™ kit, Qiagen, Co., USA). 0.2 μl of the synthesized cDNA was mixed with 3.8 μl of water, 0.5 μl of sense primer, 0.5 μl of anti-sense primer, and 5 μl of SYBR Green I staining supermix (Bio-rad, USA), which was loaded in PCR tube, followed by reaction in My IQ real-time PCR machine designed to execute reaction as follows: step 1, 95° C. for 3 minutes; step 2 (45 cycles), step 2-1, 95° C. for 10 seconds; step 2-2, 57° C. for 45 seconds; step 3, 95° C. for 1 minute; step 4, 55° C. for 1 minute; step 5 (80 cycles), 55° C. for 10 seconds. The PCR product was stained with SYBR Green I (Bio-rad, USA) to quantify thereof. SYBR Green I staining is the method taking advantage of intercalating with double-stranded DNA, and thus the more double-stranded DNA is produced, the stronger the fluorescence intensity is obtained during PCR. Primers for the target gene used for PCR and the endogenous control (MDH1) were added to SYBR Green master-mix, followed by PCR. Primer optimization was performed to determine a proper concentration. The synthesized cDNA was mixed with each primer listed in Table 5, to which SYBR Green master-mix was added. Then, PCR was performed and the result was analyzed by using quantitative software.

As a result, expression patterns of those genes [Genebank accession number NM_{—}000029 (AGT, angiotensinogen; SEQ. ID. NO: 11), Genebank accession number NM_{—}057159 (LPAR1, lysophosphatidic acid receptor 1; SEQ. ID. NO: 12), Genebank accession number NM_{—}003004 (SECTM1, secreted and transmembrane 1; SEQ. ID. NO: 13), Genebank accession number NM_{—}003810 (TNFSF10, tumor necrosis factor (ligand) superfamily, member 10; SEQ. ID. NO: 14), and Genebank accession number NM_{—}002133 (HMOX1, heme oxygenase 1; SEQ. ID. NO: 15)] were very similar to the results of microarray chip analysis of Example <2-4>. In addition, expression patterns of 5 kinds of genes which were changed by other 5 different aldehydes were investigated. As a result, the expression patterns of the genes were different from those exposed on propionaldehyde (shown either no change in expression or different expression patterns).

TABLE 5

| Genbank | | | |
|---|---|---|---|
| Accession | | PCR Primer Sequence | |
| Number | Gene Name | (5′ -> 3′) | |
| NM_000029; | AGT | Sense | TGCTGCATGGAGTGAGCAGTAGAA |
| SEQ. | (angiotensinogen) | (SEQ. ID. | |
| ID. NO: | | NO: 1) | |
| 11 | | Antisense | CACAAACAAGCTGGTCGGTTGGAA |
| | | (SEQ. ID. | |
| | | NO: 2) | |
| NM_057159; | LPAR1 | Sense | TCTGCTGGACTCCTGGATTGGTTT |
| SEQ. | (lysophosphatidic | (SEQ. ID. | |
| ID. NO: | acid | NO: 3) | |
| 12 | receptor 1) | Antisense | AAGGTGGCGCTCATTTCTTTGTCG |
| | | (SEQ. ID. | |
| | | NO: 4) | |
| NM_003004; | SECTM1 | Sense | ACTGGTGTTCAAACCCTCACCACT |
| SEQ. | (secreted and | (SEQ. ID. | |
| ID. NO: | transmembrane | NO: 5) | |
| 13 | 1) | Antisense | ATGGGTCTGCGGCATATGGAAACA |
| | | (SEQ. ID. | |
| | | NO: 6) | |
| NM_003810; | TNFSF10 | Sense | GAGCTGAAGCAGATGCAGGAC |
| SEQ. | (tumor | (SEQ. ID. | |
| ID. NO: | necrosis | NO: 7) | |
| 14 | factor | Antisense | TGACGGAGTTGCCACTTGACT |
| | (ligand) | (SEQ. ID. | |
| | superfamily, | NO: 8) | |
| | member 10) | | |
| NM_002133; | HMOX1, | Sense | ATTGCCAGTGCCACCAAGTTCAAG |
| SEQ. | hemeoxygenase 1 | (SEQ. ID. | |
| ID. NO: | | Antisense | ACGCAGTCTTGGCCTCTTCTATCA |
| 15 | | (SEQ. ID. | |
| | | NO: 10) | |

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A method for the identification of exposure to propionaldehyde comprising the following steps:
1) measuring expression levels of genes of:
Genebank accession number NM_{—}000029 (AGT, angiotensinogen; SEQ. ID. NO: 11), Genebank accession number NM_{—}057159 (LPAR1, lysophosphatidic acid receptor 1; SEQ. ID. NO: 12), Genebank accession number NM_{—}003004 (SECTM1, secreted and transmembrane 1; SEQ. ID. NO: 13), Genebank accession number NM_{—}003810 (TNFSF10, tumor necrosis factor (ligand) superfamily, member 10; SEQ. ID. NO: 14), and Genebank accession number NM_{—}002133 (HMOX1, heme oxygenase 1; SEQ. ID. NO: 15), on somatic cells separated from both an experimental group suspected with propionaldehyde exposure and a normal control group;
2) screening a subject with increased or decreased expression level by comparing the expression level of the experimental group of step 1) with that of the control group; and
3) determining the screened object of step 2) to be exposed to propionaldehyde.

2. The method for the identification of exposure to propionaldehyde according to claim 1, wherein the somatic cells of step 1) are characteristically human lung cells or human lung cancer tissue derived cells.

3. The method for the identification of specific exposure to propionaldehyde according to claim 2, wherein the human lung cancer tissue derived cells are A549.

4. The method for the identification of specific exposure to propionaldehyde according to claim 1, wherein the comparing the expression level in step 1) is performed at the level of gene or protein.

5. The method for the identification of specific exposure to propionaldehyde according to claim 4, wherein the comparing at the level of gene uses any one selected from a group consisting of RT-PCR, competitive RT-PCR, real-time RT-PCR, RNase protection assay, Northern blotting, and DNA chip.

6. The method for the identification of specific exposure to propionaldehyde according to claim 4, wherein the comparing at the level of protein uses microarray or ELISA.

7. The method for the identification of specific exposure to propionaldehyde according to claim 1, wherein the expression level of the genes of Genebank accession number NM_{—}000029 (AGT, angiotensinogen; SEQ. ID. NO: 11), Genebank accession number NM_{—}057159 (LPAR1, lysophosphatidic acid receptor 1; SEQ. ID. NO: 12), Genebank accession number NM_{—}003004 (SECTM1, secreted and transmembrane 1; SEQ. ID. NO: 13), and Genebank accession number NM_{—}003810 (TNFSF10, tumor necrosis factor (ligand) superfamily, member 10; SEQ. ID. NO: 14) is down-regulated when exposed to propionaldehyde.

8. The method for the identification of specific exposure to propionaldehyde according to claim 1, wherein the expression level of the gene of Genebank accession number NM_{—}002133 (HMOX1, hemeoxygenase 1; SEQ. ID. NO: 15) is up-regulated when exposed to propionaldehyde.

9. A method for the identification of exposure to propionaldehyde comprising the following steps:
1) extracting RNA from somatic cells obtained from both the experimental group highly suspected with propionaldehyde exposure and the normal control group;
2) synthesizing cDNA from the RNA extracted from both the experimental group and the control group of step 1), followed by labeling with different fluorescent materials;
3) hybridizing each cDNA labeled with different fluorescent materials of step 2) with a DNA microarray chip on which nucleic acid sequences of one or more genes selected from the below group or their complementary strand molecules are integrated:
Genebank accession number NM_{—}000029 (AGT, angiotensinogen; SEQ. ID. NO: 11), Genebank accession number NM_{—}057159 (LPAR1, lysophosphatidic acid receptor 1; SEQ. ID. NO: 12), Genebank accession number NM_{—}003004 (SECTM1, secreted and transmembrane 1; SEQ. ID. NO: 13), Genebank accession number NM_{—}003810 (TNFSF10, tumor necrosis factor (ligand) superfamily, member 10; SEQ. ID. NO: 14), and Genebank accession number NM_{—}002133 (HMOX1, heme oxygenase 1; SEQ. ID. NO: 15).
4) analyzing the reacted DNA microarray chip; and
5) confirming the exposure to propionaldehyde by comparing the expressions of the genes integrated on the DNA microarray chip with those of the control based on the data analyzed.

10. The method for the identification of exposure to propionaldehyde according to claim 9, wherein the somatic cell of step 1) is characteristically human lung cell or human lung cancer tissue derived cell.

11. The method for the identification of specific exposure to propionaldehyde according to claim 10, wherein the human lung cancer tissue derived cell is A549.

12. The method for the identification of exposure to propionaldehyde according to claim 9, wherein the fluorescent material of step 3) is selected from the group consisting of Cy3, Cy5, poly L-lysine-fluorescein isothiocyanate (FITC), RITC (rhodamine-B-isothiocyanate), and rhodamine.

13. A method for the identification of exposure to propionaldehyde comprising the following steps:
1) extracting RNA from somatic cells obtained from both the experimental group highly suspected with propionaldehyde exposure and the normal control group;
2) performing real-time RT-PCR (real-time reverse transcript polymerase chain reaction) with the obtained RNA using the primer sets complementary to the below genes and able to amplify them as well:
Genebank accession number NM_{—}000029 (AGT, angiotensinogen; SEQ. ID. NO: 11), Genebank accession number NM_{—}057159 (LPAR1, lysophosphatidic acid receptor 1; SEQ. ID. NO: 12), Genebank accession number NM_{—}003004 (SECTM1, secreted and transmembrane 1; SEQ. ID. NO: 13), Genebank accession number NM_{—}003810 (TNFSF10, tumor necrosis factor (ligand) superfamily, member 10; SEQ. ID. NO: 14), and Genebank accession number NM_{—}002133 (HMOX1, heme oxygenase 1; SEQ. ID. NO: 15); and
3) confirming the expression by comparing the gene product obtained in step 2) with that of the control.

14. The method for the identification of exposure to propionaldehyde according to claim 13, wherein the primer set of step 2) is characteristically composed of the forward primer and the reverse primer in the length of 18-30 mer that is able to amplify the gene of step 2).

15. The method for the identification of exposure to propionaldehyde according to claim 13, wherein the primer set of step 2) is selected from the group consisting of the following primer set 1-primer set 5:
Primer set 1: forward primer represented by SEQ. ID. NO: 1 and reverse primer represented by SEQ. ID. NO: 2;
Primer set 2: forward primer represented by SEQ. ID. NO: 3 and reverse primer represented by SEQ. ID. NO: 4;
Primer set 3: forward primer represented by SEQ. ID. NO: 5 and reverse primer represented by SEQ. ID. NO: 6;
Primer set 4: forward primer represented by SEQ. ID. NO: 7 and reverse primer represented by SEQ. ID. NO: 8; and
Primer set 5: forward primer represented by SEQ. ID. NO: 9 and reverse primer represented by SEQ. ID. NO: 10.


SEQUENCE LISTING (15 sequences)

SEQ ID NO: 1 - DNA, 24 bp, Artificial Sequence
  AGT sense primer
tgctgcatggagtgagcagtagaa

SEQ ID NO: 2 - DNA, 24 bp, Artificial Sequence
  AGT antisense primer
cacaaacaagctggtcggttggaa

SEQ ID NO: 3 - DNA, 24 bp, Artificial Sequence
  LPAR1 sense primer
tctgctggactcctggattggttt

SEQ ID NO: 4 - DNA, 24 bp, Artificial Sequence
  LPAR1 antisense primer
aaggtggcgctcatttctttgtcg

SEQ ID NO: 5 - DNA, 24 bp, Artificial Sequence
  SECTM1 sense primer
actggtgttcaaaccctcaccact

SEQ ID NO: 6 - DNA, 24 bp, Artificial Sequence
  SECTM1 antisense primer
atgggtctgcggcatatggaaaca

SEQ ID NO: 7 - DNA, 21 bp, Artificial Sequence
  TNFSF10 sense primer
gagctgaagcagatgcaggac

SEQ ID NO: 8 - DNA, 21 bp, Artificial Sequence
  TNFSF10 antisense primer
tgacggagttgccacttgact

SEQ ID NO: 9 - DNA, 24 bp, Artificial Sequence
  HMOX1 sense primer
attgccagtgccaccaagttcaag

SEQ ID NO: 10 - DNA, 24 bp, Artificial Sequence
  HMOX1 antisense primer
acgcagtcttggcctcttctatca

SEQ ID NO: 11 - DNA, 2587 bp, Artificial Sequence
  AGT sequence
atcccatgagcgggcagcagggtcagaagtggcccccgtgttgcctaagcaagactctcccctgccctctgccctctgcacctccggcctgcatgtccctgtggcctcttgggggtacatctcccggggctgggtcagaaggcctgggtggttggcctcaggctgtcacacacctagggagatgctcccgtttctgggaaccttggccccgactcctgcaaacttcggtaaatgtgtaactcgaccctgcaccggctcactctgttcagcagtgaaactctgcatcgatcactaagacttcctggaagaggtcccagcgtgagtgtcgcttctggcatctgtccttctggccagcctgtggtctggccaagtgatgtaaccctcctctccagcctgtgcacaggcagcctgggaacagctccatccccacccctcagctataaatagggcatcgtgacccggccgggggaagaagctgccgttgttctgggtactacagcagaagggtatgcggaagcgagcaccccagtctgagatggctcctgccggtgtgagcctgagggccaccatcctctgcctcctggcctgggctggcctggctgcaggtgaccgggtgtacatacaccccttccacctcgtcatccacaatgagagtacctgtgagcagctggcaaaggccaatgccgggaagcccaaagaccccaccttcatacctgctccaattcaggccaagacatcccctgtggatgaaaaggccctacaggaccagctggtgctagtcgctgcaaaacttgacaccgaagacaagttgagggccgcaatggtcgggatgctggccaacttcttgggcttccgtatatatggcatgcacagtgagctatggggcgtggtccatggggccaccgtcctctccccaacggctgtctttggcaccctggcctctctctatctgggagccttggaccacacagctgacaggctacaggcaatcctgggtgttccttggaaggacaagaactgcacctcccggctggatgcgcacaaggtcctgtctgccctgcaggctgtacagggcctgctagtggcccagggcagggctgatagccaggcccagctgctgctgtccacggtggtgggcgtgttcacagccccaggcctgcacctgaagcagccgtttgtgcagggcctggctctctatacccctgtggtcctcccacgctctctggacttcacagaactggatgttgctgctgagaagattgacaggttcatgcaggctgtgacaggatggaagactggctgctccctgatgggagccagtgtggacagcaccctggctttcaacacctacgtccacttccaagggaagatgaagggcttctccctgctggccgagccccaggagttctgggtggacaacagcacctcagtgtctgttcccatgctctctggcatgggcaccttccagcactggagtgacatccaggacaacttctcggtgactcaagtgcccttcactgagagcgcctgcctgctgctgatccagcctcactatgcctctgacctggacaaggtggagggtctcactttccagcaaaactccctcaactggatgaagaaactatctccccggaccatccacctgaccatgccccaactggtgctgcaaggatcttatgacctgcaggacctgctcgcccaggctgagctgcccgccattctgcacaccgagctgaacctgcaaaaattgagcaatgaccgcatcagggtgggggaggtgctgaacagcattttttttgagcttgaagcggatgagagagagcccacagagtctacccaacagcttaacaagcctgaggtcttggaggtgaccctgaaccgcccattcctgtttgctgtgtatgatcaaagcgccactgccctgcacttcctgggccgcgtggccaacccgctgagcacagcatgaggccagggccccagaacacagtgcctggcaaggcctctgcccctggcctttgaggcaaaggccagcagcagataacaaccccggacaaatcagcgatgtgtcacccccagtctcccaccttttcttctaatgagtcgactttgagctggaaagcagccgtttctccttggtctaagtgtgctgcatggagtgagcagtagaagcctgcagcggcacaaatgcacctcccagtttgctgggtttattttagagaatgggggtggggaggcaagaaccagtgtttagcgcgggactactgttccaaaaagaattccaaccgaccagcttgtttgtgaaacaaaaaagtgttcccttttcaagttgagaacaaaaattgggttttaaaattaaagtatacatttttgcattgccttcggtttgtatttagtgtcttgaatgtaagaacatgacctccgtgtagtgtctgtaataccttagttttttccacagatgcttgtgatttttgaacaatacgtgaaagatgcaagcacctgaatttctgtttgaatgcggaaccatagctggttatttctcccttgtgttagtaataaacgtcttgccacaataagcctccaaaaaaaa

SEQ ID NO: 12 - DNA, 3182 bp, Artificial Sequence
  LPAR1 sequence
acggcgcgctgggctcacactgtcccgccgcggacgggctttgtggttgggggcgcgcgtgcgagtgccagtgagagtgtgggtgcgcgctgtgggccgcggcgcgggtgggtggccgtgcgttcttgcgagccggcctgcaggaggcgaggctcccctggcctcccgcacccagcggcggaccgagcccctggagggaagttgccgcagccgcccgggccgccggccctcctgtcccgcgccaggtggacgtctgatttatgaagctccccatccacctatctgagtacctgacttctcaggactgacacctacagcatcaggtacacagcttctcctagcatgacttcgatctgatcagcaaacaagaaaatttgtctcccgtagttctggggcgtgttcaccacctacaaccacagagctgtcatggctgccatctctacttccatccctgtaatttcacagccccagttcacagccatgaatgaaccacagtgcttctacaacgagtccattgccttcttttataaccgaagtggaaagcatcttgccacagaatggaacacagtcagcaagctggtgatgggacttggaatcactgtttgtatcttcatcatgttggccaacctattggtcatggtggcaatctatgtcaaccgccgcttccattttcctatttattacctaatggctaatctggctgctgcagacttctttgctgggttggcctacttctatctcatgttcaacacaggacccaatactcggagactgactgttagcacatggctccttcgtcagggcctcattgacaccagcctgacggcatctgtggccaacttactggctattgcaatcgagaggcacattacggttttccgcatgcagctccacacacggatgagcaaccggcgggtagtggtggtcattgtggtcatctggactatggccatcgttatgggtgctatacccagtgtgggctggaactgtatctgtgatattgaaaattgttccaacatggcacccctctacagtgactcttacttagtcttctgggccattttcaacttggtgacctttgtggtaatggtggttctctatgctcacatctttggctatgttcgccagaggactatgagaatgtctcggcatagttctggaccccggcggaatcgggataccatgatgagtcttctgaagactgtggtcattgtgcttggggcctttatcatctgctggactcctggattggttttgttacttctagacgtgtgctgtccacagtgcgacgtgctggcctatgagaaattcttccttctccttgctgaattcaactctgccatgaaccccatcatttactcctaccgcgacaaagaaatgagcgccacctttaggcagatcctctgctgccagcgcagtgagaaccccaccggccccacagaaggctcagaccgctcggcttcctccctcaaccacaccatcttggctggagttcacagcaatgaccactctgtggtttagaacggaaactgagatgaggaaccagccgtcctctcttggaggataaacagcctccccctacccaattgccagggcaaggtggggtgtgagagaggagaaaagtcaactcatgtacttaaacactaaccaatgacagtatttgttcctggaccccacaagacttgatatatattgaaaattagcttatgtgacaaccctcatcttgatccccatcccttctgaaagtaggaagttggagctcttgcaatggaattcaagaacagactctggagtgtccatttagactacactaactagacttttaaaagattttgtgtggtttggtgcaagtcagaataaattctggctagttgaatccacaacttcatttatatacaggcttcccttttttatttttaaaggatacgtttcacttaataaacacgtttatgcctatcagcatgtttgtgatggatgagactatggactgcttttaaactaccataattccattttttcccttacataggaaaactgtaagttggaattatcttttgtttagaaagcatgcatgtaatgtatgtatgcagtatgccttacttaaaaagattaaaaggatactaatgttaaatcttctaggaaatagaacctagacttcaaagccagtatttgtttaggtcatgaagcaaacaatgctctaatcacaatattaactgtttaattaaaatgttgtaacaagtataaaacagggaatgtaagtttattaccaaagtgatatgtattccaaaaaagtcatagaagatgaagcactataatattgttcccatatatttaaaatacccaagtacattctaattaccagtatatcagaggaaaattttcgtagtctttgtaaaataatatactcatcatagaaaacttgaaaaatgcagaaatgtataaaaaagcaaaaatgattactgataatatcacaacccagaagtaaccacctttaaaaagcaacccccatgtatgcctatatgtgtattgtatactttttttacataattggagtcatactgtaaacagttttataagtagatctttttcattgcaaaattgccacattttcttatggcattaaaaattttacaaaaacataattttaatggctatattatattccatttaatggatgcaactcagtttatttaaccattcccatgttgttaactatttaggttgtttctaattttcattattataaagttgcagaaatttggtgtacataaaactgtctccatataattgattattaggatatattcccatgaaggattctttttttaaaaaaatgtgaaatgtcatcttgtacttacacctttcatgaaaagggatttcctgcttttgtactgcatgggtggcagttgtgaggaaaagccagtcaaatgacctttttacaaaagaaatgcagtggtcacttcagttgagagtgactttttaatacaacaagatcaactagaagaattcaactgtctcaagaatcaaggtaccccaatatatctcgcaattccaaactttgtttgagggactcgttatccagctcttggtagccacacctgcaatgtaaaatggaagaaaatgcaaagaaaccaaatgtgccgagtgaataaaggattgtcatatcaaaaaaaaaaaaaaaa

SEQ ID NO: 13 - DNA, 2282 bp, Artificial Sequence
  SECTM1 sequence
aaacagccccgcggggaacgcgcgccgcacgcgagctggacccgcccaggcacgaactctcctggaaaaatgctcccggcggctttcctgcttcctttagcgtgaaccgcgggtgcggtgcctcccgtgaaaataataaattcaccgtcacgcttgttgtgaacgcgggtggttcccgaaacttggaggcttcccgtaaacccagctccttcctcatctgggaggtgggtcccgcgcgggtccgccgcctcctccctggcccctccctctcgtgtctttcattttcctggggctccggggcgcggagaagctgcatcccagaggagcgcgtccaggagcggacccgggagtgtttcaagagccagtgacaaggaccaggggcccaagtcccaccagccatgcagacctgccccctggcattccctggccacgtttcccaggcccttgggaccctcctgtttttggctgcctccttgagtgctcagaatgaaggctgggacagccccatctgcacagagggggtagtctctgtgtcttggggcgagaacaccgtcatgtcctgcaacatctccaacgccttctcccatgtcaacatcaagctgcgtgcccacgggcaggagagcgccatcttcaatgaggtggctccaggctacttctcccgggacggctggcagctccaggttcagggaggcgtggcacagctggtgatcaaaggcgcccgggactcccatgctgggctgtacatgtggcacctcgtgggacaccagagaaataacagacaagtcacgctggaggtttcaggtgcagaaccccagtccgcccccgacactgggttctggcctgtgccagcggtggtcactgctgtcttcatcctcttggtcgctctggtcatgttcgcctggtacaggtgccgctgttcccagcaacgccgggagaagaagttcttcctcctagaaccccagatgaaggtcgcagccctcagagcgggagcccagcagggcctgagcagagcctccgctgaactgtggaccccagactccgagcccaccccaaggccgctggcactggtgttcaaaccctcaccacttggagccctggagctgctgtccccccaacccttgtttccatatgccgcagacccatagccgcctgcaaggcagagaggacacaggagagccagccctgagtgccgaccttgggtggcggggcctgggtctctcgtcccacccggagggcacagacaccggcttgcttggcaggctgggcctctgtgtcacccactcctgggtgcgtgcagacccttcccctccaccccccaggtcttccaagctctgcttcctcagtttccaaaatggaaccacctcacctccgcagcacccgacttaccaggacgcatgcccctccctctgccctcatcaaacccacagacccggactccctttctgccaccccaggctggtccggccccaggtgtggggtccgctctctccactcccagggctccgcgcccaagtgagggggcccctgccggagcctcagacacactccagttcagggctgtggggggccttggccacatacctgtcccttggctatgagcaggctttgggggcccttccgcggcagccccgggggccgaggtagggtcgggggcttagaggctgggatggctcctggccccaccgccagggggcagcgcaggccgggctgggaggcggcggcggcggctcgggctggggggtcaggtggacgccgccctccggggctggacgcgcatccctcagtccctcggccacccgggggtcgctccctcgtgcccaccgcacctgccgagcctctttggacccagatctgttcatgcttttgtcttcgtcactgcggcggggccctttgatgtcttcatctgtatggggtggaaaaatcaccgggaatcccccttcagttctttgaaaaagttccatgactcgaatatctgaaatgaagaaaacaaaccgactcacaaacctccaagtagctccaaatgcaatttttaaaatggaaaacaaaaatctgaaagaaacgtctttagtggctttaagccccaaaacgtccctaaggcgtcctcgagatgaagacgggggggagcccccagccaggtggagaccccgcagggacgcggcggcgcccggtgaccgaggcctcgcacagccggccgccctgagggtcgggccggagccagggtccaagaggggcgcgtttgtgtctcgggttaaaataaggttccgtccgcgtgctgggtcaga

SEQ ID NO: 14 - DNA, 1953 bp, Artificial Sequence
  TNFSF10 sequence
acagaacccagaaaaacaactcattcgctttcatttcctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagtttttttggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagagaatgaaattgctgaaagatctttcaggactctacctcatatcagtttgctagcagaaatctagaagactgtcagcttccaaacattaatgcaatggttaacatcttctgtctttataatctactccttgtaaagactgtagaagaaagagcaacaatccatctctcaagtagtgtatcacagtagtagcctccaggtttccttaagggacaacatccttaagtcaaaagagagaagaggcaccactaaaagatcgcagtttgcctggtgcagtggctcacacctgtaatcccaacattttgggaacccaaggtgggtagatcacgagatcaagagatcaagaccatagtgaccaacatagtgaaaccccatctctactgaaagtacaaaaattagctgggtgtgttggcacatgcctgtagtcccagctacttgagaggctgaggcaagagaattgtttgaacccgggaggcagaggttgcagtgtggtgagatcatgccactacactccagcctggcgacagagcgagacttggtttcaaaaaaaaaaaaaaaaaaaacttcagtaagtacgtgttatttttttcaataaaattctattacagtatgtcatgtttgctgtagtgctcatatttattgttgtttttgttttagtactcacttgtttcataatatcaagattactaaaaatgggggaaaagacttctaatctttttttcataatatctttgacacatattacagaagaaataaatttcttacttttaatttaatatga

SEQ ID NO: 15 - DNA, 1606 bp, Artificial Sequence
  HMOX1 sequence
aaatgtgaccggccgcggctccggcagtcaacgcctgcctcctctcgagcgtcctcagcgcagccgccgcccgcggagccagcacgaacgagcccagcaccggccggatggagcgtccgcaacccgacagcatgccccaggatttgtcagaggccctgaaggaggccaccaaggaggtgcacacccaggcagagaatgctgagttcatgaggaactttcagaagggccaggtgacccgagacggcttcaagctggtgatggcctccctgtaccacatctatgtggccctggaggaggagattgagcgcaacaaggagagcccagtcttcgcccctgtctacttcccagaagagctgcaccgcaaggctgccctggagcaggacctggccttctggtacgggccccgctggcaggaggtcatcccctacacaccagccatgcagcgctatgtgaagcggctccacgaggtggggcgcacagagcccgagctgctggtggcccacgcctacacccgctacctgggtgacctgtctgggggccaggtgctcaaaaagattgcccagaaagccctggacctgcccagctctggcgagggcctggccttcttcaccttccccaacattgccagtgccaccaagttcaagcagctctaccgctcccgcatgaactccctggagatgactcccgcagtcaggcagagggtgatagaagaggccaagactgcgttcctgctcaacatccagctctttgaggagttgcaggagctgctgacccatgacaccaaggaccagagcccctcacgggcaccagggcttcgccagcgggccagcaacaaagtgcaagattctgcccccgtggagactcccagagggaagcccccactcaacacccgctcccaggctccgcttctccgatgggtccttacactcagctttctggtggcgacagttgctgtagggctttatgccatgtgaatgcaggcatgctggctcccagggccatgaactttgtccggtggaaggccttctttctagagagggaattctcttggctggcttccttaccgtgggcactgaaggctttcagggcctccagccctctcactgtgtccctctctctggaaaggaggaaggagcctatggcatcttccccaacgaaaagcacatccaggcaatggcctaaacttcagagggggcgaagggatcagccctgcccttcagcatcctcagttcctgcagcagagcctggaagacaccctaatgtggcagctgtctcaaacctccaaaagccctgagtttcaagtatccttgttgacacggccatgaccactttccccgtgggccatggcaatttttacacaaacctgaaaagatgttgtgtcttgtgtttttgtcttatttttgttggagccactctgttcctggctcagcctcaaatgcagtatttttgttgtgttctgttgtttttatagcagggttggggtggtttttgagccatgcgtgggtggggagggaggtgtttaacggcactgtggccttggtctaacttttgtgtgaaataataaacaacattgtctgatagtagcttgaaaaaaaaaaaaaaaaaa